SEQUENCE LISTING (7 sequences)

SEQ ID NO: 1 - protein, 122 aa, Homo sapiens
RCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNP

SEQ ID NO: 2 - protein, 122 aa, Mus musculus
RCKDDEFTHLYTLIVRQDNTYEVKIDNSQVESGSLEDDGDFLPPKKIKDPDAAKPEDWDERAKIDDPTDSKPEDWDKPEHIPDPDAKKPEDWDEEMDGEWEPPVIQNPEYKGEWKPRQIDNP

SEQ ID NO: 3 - protein, 122 aa, Rattus norvegicus
RCKDDEFTHLYTLIVRPDNTYEVKIDNSQVESGSLEDDWDFLPPKKIKDPDAAKPEDWDERAKIDDPTDSKPEDWDKPEHIPDPDAKKPEDWDEEMDGEWEPPVIQNPEYKGEWKPRQIDNP

SEQ ID NO: 4 - DNA, 366 bp, Homo sapiens
cgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaaccca

SEQ ID NO: 5 - DNA, 366 bp, Mus musculus
cggtgtaaggatgatgaattcacacacctatacacactgattgtgcggcaagacaacacctatgaggtgaaaattgacaacagccaggtggagtcaggctccttggaggatgatggggactttctgccacccaagaagataaaggaccctgatgctgccaagccggaagactgggatgaacgagccaagatcgatgaccccacagattccaagcctgaggactgggacaagccagagcacatccctgaccctgatgctaagaagcctgaggactgggatgaagagatggatggagagtgggaaccaccagtgattcaaaatcctgaatacaagggcgagtggaaaccacgtcaaattgacaaccca

SEQ ID NO: 6 - DNA, 366 bp, Rattus norvegicus
cggtgtaaggatgatgaattcacacatctatacacgctgattgtgcggccagacaacacctacgaggtgaaaattgacaacagccaggtggagtcgggctccttggaggatgattgggactttctgccgcccaagaagattaaggatcctgacgctgccaagccagaagactgggatgaacgagccaagattgatgaccccacagattccaagcctgaggactgggacaagccagagcacatccctgaccctgatgctaagaagcctgaggactgggacgaagagatggatggagagtgggaaccaccagtgattcaaaatcctgaatacaagggcgaatggaagccacgtcaaattgacaaccca

SEQ ID NO: 7 - protein, 417 aa, Homo sapiens
MLLSVPLLLGLLGLAVAEPAVYFKEQFLDGDGWTPRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

(19) United States
(12) Patent Application Publication
(10) Pub. No.: US 2004/0259787 A1
(43) Pub. Date: Dec. 23, 2004

(54) C1q and collectin receptor

(76) Inventor: Wilhelm Schwaeble, Leicester (GB)

Correspondence Address: Monique A. Morneault, Esq., Wallenstein Wagner & Rockey, Ltd., 53rd Floor, 311 South Wacker Drive, Chicago, IL, 60606-6630, US

(21) Appl. No.: 10/744,936

(22) Filed: Dec. 23, 2003

Related U.S. Application Data
(63) Continuation of application No. 09/503,421, filed on Feb. 14, 2000, now abandoned.
Application No. 09/503,421 is a continuation of application No. PCT/GB98/02430, filed on Aug. 12, 1998.

(30) Foreign Application Priority Data
Aug. 12, 1997 (GB) 9716998.1

(51) Int. Cl. A61K 38/56

(52) U.S. Cl. 514/12

(57) ABSTRACT

The present invention concerns novel uses of the cC1q Receptor (cC1qR) binding domain and inhibitors thereof.

This is a continuation application of co-pending U.S. patent application Ser. No. 09/503,421, filed Feb. 14, 2000, which is a continuation of PCT/GB98/02430 filed Aug. 12, 1998 (claiming priority from British Appln. No. 9716998.1, filed Aug. 12, 1997), now pending, which is hereby incorporated by reference.

The present invention concerns novel uses of the C1q and collectin Receptor (cC1qR) binding domain and inhibitors thereof.

The C1q binding domain within cC1qR (cC1q receptor) has previously been identified (Stuart, G. R. et al., 1996, FEBS Letters, 397: 245-249 and references therein). The cC1q receptor is also be referred to as the C1q receptor, which also binds collecting. The cC1qR homologue Calreticulin (CaR/CRT) has also been identified and shown to have very high sequence homology, and as such reference to cC1qR is considered to also be reference to CRT and vice versa unless otherwise stated.

Certain functionality has been attributed to cC1qR upon its activation by the binding of C1q, namely immunological responses such as phagocytosis, enhanced cytokine and antibody production and antibody-dependent cell cytotoxicity. cC1qR is also known to bind the collectin proteins SP-A, MBL, CL43 and conglutinin. However, the exact nature of cC1qR has not yet been determined nor its structure identified. Sequence analysis does not identify it as being part of a known class of cell-surface receptors.

The present inventors have now found that the C1q binding domain of cC1qR is in fact a CUB (Complement Ubiquitin) domain, and as such certain previously unknown functionality can be attributed to cC1qR and inhibitors of same. CUB domains are well known (see for example Day, A. J. et al., 1993, Behring Inst. Mitt., 93: 31-40; Thiel, S. et al., 1997, Nature, 386: 506-510; Arlaud, G. J. et al., 1993, Behring Inst. Mitt., 93: 189-195).

Thus according to the present invention there is provided the use of a cC1qR binding domain as a CUB domain. Thus the present invention may provide the use of a cC1qR binding domain in the manufacture of a medicament to effect CUB domain functionality. Since the cC1qR binding domain is both a collectin and C1qR binding domain, the term “cC1qR binding domain” also encompasses C1q binding domains.

Sequence analysis of cC1qR shows little primary homology with known CUB domains, but the cC1qR binding domain does in fact have 6 to 7 consensus residues (out of a total of about 100) with CUB and this provides the cC1qR binding domain (and thus cC1qR) with CUB domain functionality.

The C1q binding domain may form part of an existing molecule, for example cC1qR or it may form part or the whole of a novel molecule, for example a molecule comprising a recombinant cC1qR binding domain. The cC1qR binding domain may bind a site comprising five collagen repeats (Gly-X-Y triplets) (Malhotra, R. et al., Biochem. J., 293: 15-19).

Also provided according to the present invention is the use of an inhibitor of the cC1qR binding domain to inhibit CUB functionality. Such an inhibitor may of course be any molecule or other chemical agent which is capable of inhibiting the activation of the cCl q receptor. Examples of such inhibitors include recombinant cC1qR binding domains which competitively inhibit the binding of C1q to cC1 qR and thereby inhibit the activation of the cC1 q receptor.

Such competitive inhibitors (a) inhibit C1q and mannose binding lectin mediated activation of complement and (b) bind to the part of the collagenous region of C1q where covalent binding to ss-amyloid occurs in Alzheimer's disease.

Also provided according to the present invention is a method of manufacture of a medicament to effect CUB domain functionality, characterised in the use of a cC1qR binding domain. Also provided according to the present invention is a method of manufacture of a medicament for inhibiting CUB domain functionality, characterised in the use of an inhibitor of the cC1qR binding domain.

The identification of the cC1qR binding domain as a CUB domain provides a wide range of previously unidentified functionality for the cC1q receptor and inhibitors thereof. Inhibition of the cC1qR binding domain prevents (i.e. inhibits) complement activation via the classical and lectin pathways and provides therapeutic potential in all such diseases in which complement activation is involved in the initiation and maintenance of inflammation, for example myocardial infarction, brain ischemia (stroke), gut ischemia, rheumatoid arthritis, systemic lupus erythematosus, burns and immune complex nephritis. The cC1qR binding domain may also be used to inhibit the binding of β-Amyloid to C1q, thereby inhibiting the formation of amyloid plaques in Alzheimers disease (Velazquez, P. et al., 1997, Nat. Med., 3(1): 77-79). Additional CUB functionality includes the ability to bind carbohydrate domains of molecules, for example of collagens, and to cause opsonisation.

Thus the present invention also provides the use of an inhibitor of the cC1qR binding domain according to the present invention in the manufacture of a medicament for the treatment of complement activation involved in the initiation and maintenance of inflammation, for example for the treatment of myocardial infarction, brain ischemia (stroke), gut ischemia, rheumatoid arthritis, systemic lupus erythematosus, burns, or immune complex nephritis. Also provided is the use of an inhibitor of the C1qR binding domain according to the present invention in the manufacture of a medicament for the treatment of amyloid plaques in Alzheimer's disease.

The present invention also provides the advantage of achieving the desired effecting or inhibiting of CUB domain functionality through the use of a low molecular weight molecule.

Various cC1qR binding domains have been identified by the present inventors, namely those of humans (SEQ ID NO: 1), mice (SEQ ID NO: 2) (Mus musculus C57 black) and rats (SEQ ID NO: 3) (Rattus norvegicus whistar). Thus the cC1qR binding domain may have the sequence of any one of SEQ ID NOS: 1-3. Obviously, the sequence may be partially modified to retain CUB domain functionality yet have a sequence which is different from the one from which it was derived, i.e. one of SEQ ID NOS: 1-3, and the present invention encompasses the use of such partially modified domains. Partial modification may, for example, be by way of addition, deletion or substitution of amino acid residues. Substitutions may be conserved substitutions. Hence the partially modified molecule may be a homologue of the molecules from which it was derived. It may, for example, have at least 40% homology with the molecule from which it was derived. It may for example have at least 50, 60, 70, 80, 90 or 95% homology with the molecule from which it was derived. An example of a homologue is an allelic mutant.

Also provided according to the present invention is a method of treatment of the human or animal body comprising the use of a cC1qR binding domain or an inhibitor thereof according to the present invention. The method may be a method for effecting (in the case of a cC1qR binding domain) or the inhibiting (in the case of a cC1qR binding domain inhibitor) CUB domain functionality.

The invention will be further apparent from the following description, with reference to the several figures of the accompanying drawings, which show, by way of example only, forms of cC1qR binding domain. Of the figures:

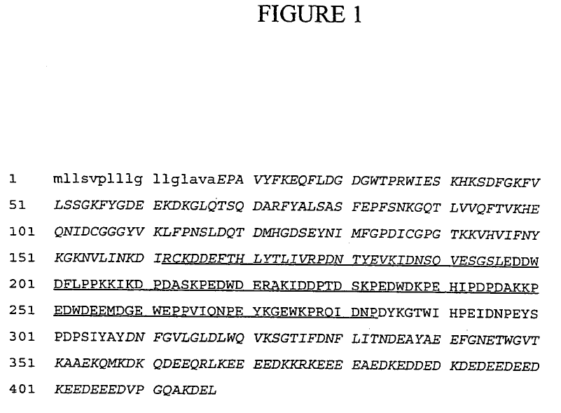

FIG. 1 shows the amino acid sequence of CRT (SEQ ID NO: 7). Amino acid sequences were deduced from the nucleotide sequence of human CRT (McCauliffe, D. P. et al., 1990, J. Clin. Invest., 85: 1379-1391.). The signal sequence residues are shown in lower case. The N-(italics), P- and C-(italics) domains are indicated. The CUB-domain is underlined. Domain constructs were expressed as thioredoxin fusion products;

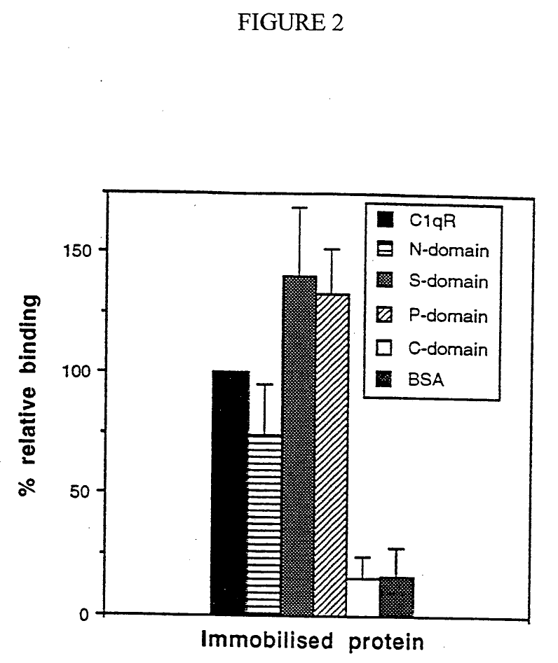

FIG. 2 shows binding of C1q to Calreticulin domains. Solid-phase bound domains, with appropriate controls, were incubated with radioiodinated C1q. Binding levels of four separate experiments, at saturation, are shown, calculated as % (bound/loaded). These percentages were then standardised against the results for cC1qR. Y-axis shows % relative binding. X-axis shows immobilised proteins (left to right): C1qR, N-domain, S-domain, P-domain, C-domain and BSA;

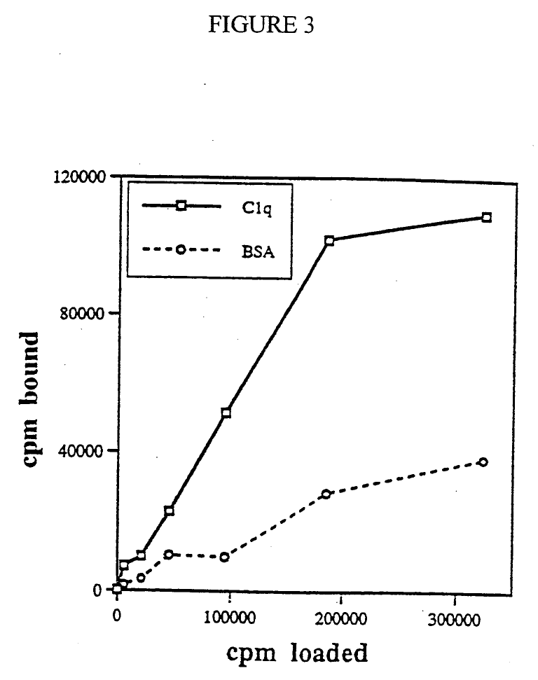

FIG. 3 shows binding of CUB-domain to immobilised C1q. Serial dilutions of radiolabelled CUB-domain were bound to immobilised C1q and BSA. After extensive washing, bound radioactivity was measured as described in Experimental section (below). Y-axis shows cp1 bound. X-axis shows cpm loaded. Upper line (solid, with open squares) is C1q. Lower line (dashed, with open circles) is BSA;

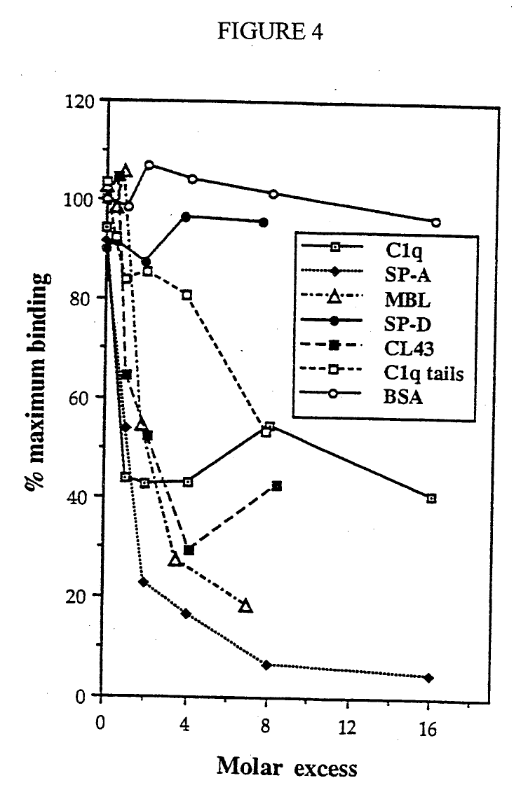

FIG. 4 shows the inhibition of CUB-domain-C1q interaction by collecting, C1q and C1q collagen tails. Constant levels of radiolabelled CUB-domain were pre-incubated with serial dilutions of unlabelled C1q, C1q tails and collectin proteins. The incubation mixture was bound to, and eluted from, solid phase C1q. Solid line with open squares is C1q; dashed line with diamonds is SP-A; dashed line with open triangles is MBL; solid line with solid circles is SP-D; dashed line with solid squares is CL43; dashed line with open squares is C1q tails; solid line with open circles is BSA; and

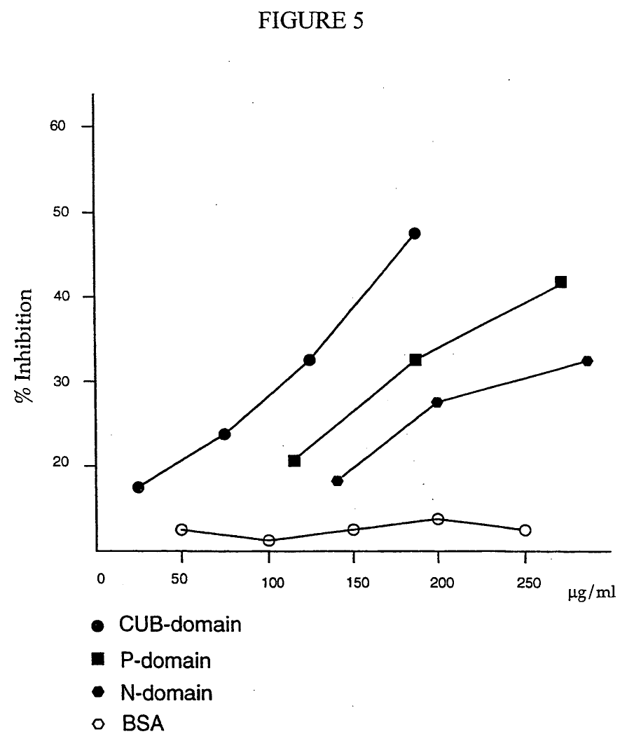

FIG. 5 shows the inhibition of the classical pathway of complement activation by addition of the recombinant CUB domain of cC1qR. Haemolytic assays were used to determine the concentration-dependence of the inhibition of the classical activation pathway of complement by the recombinant CUB domain, the N-domain and the P-domain of cC1qR. Y-axis shows % inhibition. X-axis hows μg/ml. Line with solid circles is CUB-domain. Line with solid squares is P-domain. Line with solid hexagons is N-domain. Line with open circles is BSA.

### EXPERIMENTAL

The following experiments demonstrate the activity of the C1q binding domain of cC1qR and that the C1q binding domain of cC1qR is in fact a CUB and show (see FIG. 5) the inhibition of the classical pathway of complement activation by addition of the recombinant CUB domain of cC1qR.

The sequence listing includes: the DNA sequence (SEQ ID NO: 4) and derived amino acid sequence (SEQ ID NO: 1) of human calreticulin; the DNA sequence (SEQ ID NO: 5) and derived amino acid sequence (SEQ ID NO: 2) of mouse calreticulin; the DNA sequence (SEQ ID NO: 6) and derived amino acid sequence (SEQ ID NO: 3) of rat calreticulin; and the amino acid sequence of CRT (SEQ ID NO: 7). Amino acid sequences were deduced from the nucleotide sequence of human CRT (McCauliffe, D. P. et al., 1990, J. Clin. Invest., 85: 1379-1391.). The signal sequence residues of SEQ ID NO: 7 are residues 1-17. The N-domain (shown in italics) is residues 18-196; the P-(residues 197-308) and C-(residues 309-417) (italics) domains are indicated. The CUB-domain is residues 162-283 (underlined). Domain constructs were expressed as thioredoxin fusion products.

Recombinant CRT domains to be tested for C1q and collectin binding function were produced from a cDNA clone (phCRT-1) isolated from a human umbilical vein endothelial cell cDNA library (Stuart, G. R. et al., 1996, Exp. Lung Res., 22: 467-487; Stuart, G. R. et al., 1996, FEBS Lett., 397: 245-249). These domains, as described below, are based upon structural predictions for the molecule and have previously been used to localise CRT function within the molecule. The amino-terminal N-domain contains the binding regions for PDI (Baksh, S. et al., 1995, J. Biol. Chem., 270(52): 31338-31344), Zn²⁺ (Baksh, S. et al., 1995, FEBS Lett., 376(1-2): 53-57) and integrins (Leung-Hagesteijn, C. Y. et al., 1994, J. Cell Sci., 107 (Pt 3): 589600). The proline-rich central P-domain contains the high affinity Ca²⁺ binding site (Baksh, S. and Michalak, M., 1991, J. Biol. Chem., 266: 21458-21465) and the lectin site (D. Williams, cited in Krause, K. H. and Michalak, M., 1997, Cell, 88(4): 439-443) within two sets of highly conserved repeats. The acidic C-domain contains the ER-retention terminal KDEL signal (McCauliffe, D. P. et al., 1990, J. Clin. Invest., 85: 1379-1391) and the low affinity Ca²⁺ binding site (Baksh, S. and Michalak, M., 1991, J. Biol. Chem., 266: 21458-21465). Previous studies have indicated that the C1q binding site lies across the intersection of the N and P-domains (Stuart, G. R. et al., 1996, FEBS Lett., 397: 245-249; Stuart, G. R. et al., 1997, Immunopharmacology, 38:73-80). Within this region we have identified and expressed a 123 amino acid region containing a putative C1r/C1s (also termed CUB) module (Day, A. J. et al., 1993, Behring Inst. Mitt., 93: 31-40) based upon amino acid sequence alignments. We termed this segment the CUB-domain and show here that it contains the C1q and collectin-binding site of cC1qR/CRT.

Purification and Radioiodination of cC1qR, C1q and Collectins

Native cC1qR was purified from human U937 cells as previously described (Malhotra, R. et al., 1993, Immunology, 78: 341-348). cC1qR and CUB-domain samples were iodinated by the Iodogen method (Fraker, P. J. and Speck, J. C. Jr., 1978, Biochem. Biophys. Res. Commun., 80: 849-857). C1q was purified as previously described (Reid, K. B. M., 1981, Methods in Enzymology, 80: 16-25) and radioiodinated as described by Bolton & Hunter (Bolton, A. E. and Hunter, W. M., 1973, Biochem. J., 133: 529-539) as this method of iodination causes less damage to large, oxidation-sensitive molecules such as C1q than the more frequently utilised Iodogen method (Stuart, G. R. et al., 1996, Exp. Lung Res., 22: 467-487). Radiolabelled proteins were stored at 4° C. C1q collagen tails were prepared as described by Reid (Reid, K. B. M., 1976, Biochem. J., 155: 5-17). Collectins were purified as previously described (Malhotra, R. et al., 1990, J. Exp. Med., 172: 955-959; Holmskov, U. et al., 1995, Biochem. J., 305: 889-896).

Prokaryotic Expression of Recombinant Calreticulin Domains

Given that CRT, C1r and C1s all interact with C1q, a sequence comparison was performed to investigate the structural basis for this interaction. A region that may correspond to a CUB module was identified in CRT and was analysed by multiple sequence alignment as described previously (Day, A. J. et al., 1993, Behring Inst. Mitt., 93: 31-40). This region, termed the S region (C1s-like (CUB) domain), spans the intersection of the N and P-domains (residues 160-283). A 1.9 kb cDNA clone for CRT (phCRT-1) was isolated from a human umbilical vein endothelial cell library in the eukaryotic expression vector CDM8 (Aruffo, A. and Seed, B., 1987, PNAS USA, 84: 8573-8577). Sequence analysis revealed that phCRT-1 comprised the complete coding sequence for CRT with absolute identity to the previously published human CRT sequence (McCauliffe, D. P. et al., 1990, J. Clin. Invest., 85: 1379-1391).

The Thiobond expression system was used to produce N, P, C and CUB-domains of cC1qR/CRT (representing the N-terminal region, the proline-rich central region, the C-terminal region, and a region spanning the intersection of the N and P-domains (as described above) (FIG. 1). The individual domains were expressed as thioredoxin fusion proteins in E. Coli using the plasmid pTrxfus (Invitrogen BV, Leek, Netherlands) as described previously (Stuart, G. R. et al., 1996, FEBS Lett., 397: 245-249; Stuart, G. R. et al., 1997, Immunopharmacology, 38: 73-80).

Samples were assayed for recombinant calreticulin domain expression by SDS-PAGE (Laemmli, U. K., 1970, Nature, 227: 680-685) and by Western blotting with rabbit antisera to:(1) whole cC1qR (raised against human cC1qR purified from U937 cells (Malhotra, R. et al., 1993, Immunology, 78: 341-348); (2) CRT C-terminal region (raised against a GST fusion protein containing the final 18 residues of recombinant human CRT), and (3) CRT N-terminal region (raised against a GST fusion protein containing residues 7-18 of recombinant human CRT).

Interaction of Immobilised Recombinant Human cC1qR/CRT Domains with Radiolabelled C1q

Binding experiments with the cC1qR/CRT domains were performed throughout in low salt (10 mM potassium phosphate, 0.5 mM EDTA (pH 7.4)) in order to maximise the ionic interaction with C1q.

Microtitre plates were coated with the N-, P-, C- and CUB-domains and with three controls, cC1qR, BSA and Thioredoxin, (8 mg/ml in 35 mM NaHCO3, 15 mM Na2CO3 pH 9.6) for 2 hr at 37° C. Non-specific interactions were blocked by incubation with 10 mM potassium phosphate, 0.5 mM EDTA pH 7.4 containing BSA (10 mg/ml). Any free —SH groups in the samples, due to the presence of the thioredoxin fusion protein, were blocked by a brief washing step using the phosphate buffer containing 2 mM iodoacetamide. After washing, serial dilutions of radioiodinated C1q (in 10 mM potassium phosphate, 0.5 mM EDTA, pH 7.4) were added to the wells and incubated for 2 h at 37° C. Wells were washed three times with phosphate buffer and bound radioactivity eluted with 100 ml 4M NaOH and measured.

Concentration Dependent Binding of Radiolabelled CUB-Domain to Immobilised C1q C1q Binds to the Fc Regions of IgG.

This property was utilised in order to correctly orient the C1q on microtitre plates. Breakable microtitre plates (Life Sciences International) were coated with rabbit Fc (5 mg per well in 35 mM NaHCO₃, 15 mM Na₂CO₃ pH 9.6). Non-specific sites were blocked as described above, and the wells were incubated with C1q (5 mg per well in 10 mM potassium phosphate buffer). Certain wells were also coated with BSA as a negative control. After further washing, serial dilutions of radioiodinated CUB-domain (in 10 mM potassium phosphate, 0.5 mM EDTA, pH 7.4) were added to the wells and incubated for 2 h at 37° C. Wells were washed three times with the phosphate buffer and bound radioactivity in the individual wells measured.

Competitive Inhibition of the CUB-domain-C1q Interaction by Fluid Phase C1q, Collectins and C1q Collagen Tails

C1q was immobilised onto Fc-coated microtitre plates as described above. Non-specific binding was blocked by incubation with 10 mM potassium phosphate, 0.5 mM EDTA (pH 7.4) containing BSA (10 mg/ml). Serial dilutions of the collectins (SP-A, MBL, SP-D, CL43), C1q, C1q tails and BSA (maximum quantity=9 mg/well) were prepared in 10 mM potassium phosphate, 0.5 mM EDTA (pH 7.4). Each dilution (100 ml) was then incubated for 1 h at 37° C. with a constant level of radiolabelled CUB-domain and loaded onto the plate. Following 2 hours incubation at 37° C., wells were extensively washed and bound radioactivity measured.

Results

Amino Acid Sequence Alignments

A region spanning the intersection of the N- and P-domains of CRT has previously been implicated in C1q binding (Stuart, G. R. et al., 1996, FEBS Lett., 397: 245-249; Stuart, G. R. et al., 1997, Immunopharmacology, 38: 73-80). Amino acid sequence alignment of a region within CRT (residues 160-283) showed little primary homology to a CUB module, but the region is in fact a CUB module. Two CUB modules, together with an EGF module, form a binding region within C1r2C1s2 for the collagenous tails of C1q. cC1qR competes with C1r2C1s2 for binding to C1q, implying a similarity in C1q binding sites on C1r, C1s and cC1qR (Sobel, A. T. and Bokisch, V. A., in: Membrane receptors of lymphocytes (M. Seligman, Fla. Preud'homme, FM Kourilsky eds). North Holland Publishing Co., Amsterdam, pll, 1975; Van den Berg, R. H. et al., 1995, Eur. J. Immunol., 25(8): 22062210). This segment, the CUB-domain (residues 162-283, see FIG. 1), was tested for C1q binding.

Interaction of Recombinant Human Calreticulin Domains with C1q N-, P-, C-, and CUB-domains of human CRT were expressed as thioredoxin fusion proteins. Correct expression was verified by SDS-PAGE and Western blotting. FIG. 2 summarises the results of four separate solid phase direct binding experiments. Significant binding to radioiodinated C1q was observed for cC1qR, the CUB-domain, the P-domain, and, to a lesser extent, the N-domain. The C-domain showed no binding. Serial dilutions of radioiodinated CUB-domain were incubated with immobilised C1q and BSA (FIG. 3). Concentration-dependent, saturable binding was observed to C1q but not to BSA.

Competitive Inhibition of C1q-CUB-Domain Interaction by C1q Tails and Collectins.

C1q was immobilised on microtitre plates by interaction with solid phase Fc. FIG. 4 shows the results of competitive inhibition of the CUB-domain-C1q interaction. As expected, native fluid-phase C1q demonstrates concentration-dependent inhibition. C1q tails also cause inhibition, indicating that the interaction of the CUB-domain with C1q is via the collagenous C1q tails. Inhibition studies with the collectin proteins demonstrated that SP-A, MBL and CL43 interact with the CUB-domain, via the same, or an overlapping binding site as C1q. SP-D and BSA did not inhibit the CUB-domain-C1q interaction.

Competitive Inhibition of C1q Mediated Complement Activation, i.e. Classical Pathway Activation.

The ability of cC1qR and fragments thereof to bind to C1q and interfere with binding of the C1q associated serine proteases C1r2C1s2 was determined by haemolysis assays. Sheep erythrocytes (E) and rabbit anti-sheep erythrocytes (A) were used to prepare EAC1q cells as described by Borsos and Rapp (Borsos, T. and Rapp, H. J., 1967, J. Immunol., 99: 263-268) and de Bracco and Stroud (De Bracco, M. M. and Stroud, R. M., 1971, J. Clin. Invest. 50: 838-848). EA and EAC1q cells (100 ml×10⁸ cells/ml) were incubated at 37° C. for 1 h with serial dilutions of C1q deficient serum, from a patient with homozygous C1q deficiency, in order to establish the minimum serum concentration required to cause complete cell lysis od EAC1q cells. EAC1q cells (100 ml) were then incubated for 1 h at 37° C. with increasing concentrations of the recombinant N-domain, P-domain and the CUB-domain of human cC1qR and BSA as a control and after incubation, the extent of lysis established by the addition of cold DGVB++(1 ml) (Mayer, 1961). After centrifugation (3000 g, 10 min) the OD⁴¹² of the supernatant was measured. Controls of 100% lysis comprise 100 ml cells and 1.1 ml water. The results of this assay are shown in FIG. 5. Hereby, the CUB domain of cC1qR was shown to effectively inhibit C1q mediated complement activation. At higher concentrations, the P-domain and the N-domain (which contain parts of the CUB-domain) show inhibition at a lesser extent.

1. The use of a cC1qR binding domain in the manufacture of a medicament to effect CUB domain functionality.

2. The use of a cC1qR binding domain according to claim 1, the binding domain forming part of a C1q receptor.

3. The use of a cC1qR binding domain according to either one of claims 1 or 2, the cC1qR binding domain being recombinant.

4. The use of an inhibitor of the cC1qR binding domain in the manufacture of a medicament to inhibit CUB functionality.

5. The use of an inhibitor of the cC1qR binding domain according to claim 4, the inhibitor comprising a cC 1 qR binding domain.

6. The use of an inhibitor of the cC1qR binding domain according to either one of claims 4 or 5 in the manufacture of a medicament for the treatment of complement activation involved in the initiation and maintenance of inflammation.

7. The use of an inhibitor of the cC1qR binding domain according to claim 6 in the manufacture of a medicament for the treatment of myocardial infarction, brain ischemia (stroke), gut ischemia, rheumatoid arthritis, systemic lupus erythematosus, burns, or immune complex nephritis.

8. The use of an inhibitor of the cC1qR binding domain according to either one of claims 4 or 5 in the manufacture of a medicament for the treatment of amyloid plaques in Alzheimer's disease.

9. The use of a cC1qR binding domain or inhibitor thereof according to any one of the preceding claims, the cC1qR binding domain having the sequence of any one of SEQ ID NOS: 1-3 or a partially modified form thereof.

10. A method of treatment of the human or animal body comprising the use of a cC1qR binding domain or an inhibitor thereof according to any one of the preceding claims.
